(19) United States
(12) Patent Application Publication
(10) Pub. No.: US 2009/0169704 A1
(43) Pub. Date: Jul. 2, 2009

(54) Process for the enzymatic preparation of a gamma-glutamyl compound

(75) Inventors: Lokesh Basavaraju, Bangalore (IN); Payal Gupta, Bangalore (IN); Sreeramulu Guttapadu, Bangalore (IN); Matthew John, Bangalore (IN); Vilas Pandurang Sinkar, Bangalore (IN); Narayanaswami Subramanian, Bangalore (IN); Purna Venkatesh, Bangalore (IN)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc. d/b/a Unilever

(21) Appl. No.: 12/316,962

(22) Filed: Dec. 18, 2008

(30) Foreign Application Priority Data
Dec. 28, 2007 (IN) 2601/MUM/2007
Apr. 11, 2008 (EP) EP08154377

(51) Int. Cl. A23F 3/00; C12P 13/14; C07C 229/00

(52) U.S. Cl. 426/597; 435/110; 562/561

(57) ABSTRACT

Disclosed is a process for the enzymatic preparation of a gamma-glutamyl compound. The process comprises a step of contacting a gamma-glutamyl donor and a gamma-glutamyl acceptor with an aqueous medium comprising a gamma glutamyl transpeptidase enzyme. The enzyme is derived from a plant belonging to the Graminaceae or Leguminaceae family, or from Camellia sinensis.

### TECHNICAL FIELD

The invention relates to a process of enzymatic preparation of gamma glutamyl compounds. It particularly relates to processes using a gamma glutamyl transpeptidase enzyme.

### BACKGROUND OF THE INVENTION

Any discussion of the prior art throughout the specification should in no way be considered as an admission that such prior art is widely known or forms part of the common general knowledge in the field.

Gamma glutamyl transpeptidase (GGT, EC 2.3.2.2) is a heterodimeric enzyme. It catalyzes the cleavage of gamma glutamyl compounds such as glutathione and the transfer of their gamma glutamyl moiety to other amino acids and peptides. It is found in bacteria, mammals and plants. However, the enzymes from various sources do not have structural homology. Gamma glutamyl transpeptidase has been isolated from kidney bean fruit, radish, onion, tomato, and transgenic tobacco. The optimum pH of kidney bean GGT is around 9.5, that of onion is around 9. In the case of GGT obtained from tomato, the gamma-glutamyl transpeptidation activity has been reported to be optimum at pH between 8 and 9.5. However the activity drops to near zero between pH of 5.5 and 6.5. In case of GGT obtained from transgenic tobacco, although hydrolytic activity of GGT was reported to be over a broad pH range, the gamma-glutamyl transpeptidation activity was not reported. In case of GGT obtained from radish, optimum pH was reported to be about 7.5 for soluble GGT (GGT I and GGT II) and about 7.5-8 for bound GGT (GGT A and GGT B). However, the activity for soluble GGT was reported to drop to 50% at a pH of 7. Further, use of GGT obtained from plant material with ethylamine as acceptor for synthesis of theanine has not been reported.

The process of gamma glutamylation is of commercial importance for several reasons. Water solubility of some amino acids and peptides can be significantly improved by gamma glutamylation. Several gamma-glutamyl compounds, such as L-theanine (gamma-glutamyl ethyl amide) are known to have beneficial effects on health. Similarly, gamma glutamylation can improve taste and organoleptic properties of certain amino acids and peptides, particularly those with a bitter taste.

L-theanine (Gamma-glutamyl ethyl amide) is a non-protein amino acid naturally occurring mainly in tea. Typically theanine is present in an amount of from 0.1 to 2% by dry weight of tea leaf. In recent years, theanine has been shown to have several health benefits including reducing anxiety related disorders, lowering blood pressure, reducing post-menopausal syndromes, improving immunity, increasing attention etc, and consequently, there have been several attempts to prepare theanine.

Chemical synthesis of theanine is a relatively complex multistep process. Theanine has also been prepared by extraction of tea leaves and by use of tea callus. These methods have problems related to low yield and/or purity, and are difficult to scale up.

Synthesis of theanine using microbial enzymes has been reported by several researchers. Suzuki et al (Enzyme Microbiol. Tehnol. 31, 884-889, 2002) have reported synthesis of theanine using ethylamine and glutamine as substrates and bacterial gamma glutamyl transferase. Suzuki et al (Amino Acids, Vol 32, p333-340, 2007) have reviewed the literature on enzymatic preparation of gamma-glutamyl compounds using bacterial gamma glutamyl transpeptidase. In the processes using pathogenic bacteria, it is necessary to ensure that the final product is free from microbial contamination. Further, the microbes, even if non-pathogenic, may produce toxins and other metabolic products which must be separated from the final product, adding to complexity and cost. The optimum pH of these processes is around 9.5 and the range of operating pH is relatively narrow. It is particularly difficult to obtain good yield of theanine at pH of around 7.5 or lower. This makes it difficult to integrate the process of preparation of theanine in tea-processing. Tea has a natural pH close to 5 and addition of material with a different pH has a negative impact on the organoleptic properties of tea. Even if the pH is adjusted with acidulants, the acidulants in turn change the organoelptic properties. Further, the cost of theanine prepared using the known processes is relatively high due to high cost of enzyme derived from microbial and/or animal source. In the case of microbial enzyme, additional process steps such as sterilization and separation of toxins increase the cost.

GGT has also been obtained from mammals. Porcine or bovine kidney GGT is commercially available. It is however relatively expensive and extensive purification and separation steps are required before using an enzyme derived from animals for treatment of food products. Further, GGT derived from animals has an optimum pH of around 9.5.

Masi et al (J. Plant. Phisiol, Vol. 164, pp. 1527-1535, 2007) have reported localization of GGT in Zea mays organs and tissues.

Kawasaki et al (Bichimica et Biophysica Acta, Vol. 716, pp. 194-200, 1982) have reported a GGT from pea seedlings wherein the maximum activity of the reaction between L-glutamine and D-alanine was observed at pH 9.5.

Sasaoka et al (Agr. Biol. Chem., Vol. 29, No 11 p 984-988, 1965) reported synthesis of theanine using enzymes recovered from acetone extract of tea seedlings, pea seedlings and pigeon liver. The enzymatic preparation of theanine using acetone extract of pea and pigeon liver was ascribed to non-specific reaction of glutamine synthetase whilst the preparation using enzymes isolated from tea seedlings was attributed to another synthetase which the authors named as L-glutamyate:ethylamine ligase. The synthesis reported therein was attributed to synthetase type enzymes wherein presence of energy source ATP and cofactors such as magnesium or manganese is essential.

Therefore, one of the objects of the present invention is to overcome or ameliorate at least some of the disadvantages of the prior art or to provide a useful alternative.

Another object of the present invention is to provide a process of synthesis of gamma-glutamyl compounds that can be carried out over a relatively broad range of pH.

Another object of the present invention is to provide a process of synthesis of gamma glutamyl compounds that can be carried out at pH less than 8.5, more preferably, less than 7.

Another object of the present invention is to provide a process of synthesis of gamma-glutamyl compounds that can be carried out over a relatively broad range of temperature.

Another object of the present invention is to provide a process for preparation of theanine that can utilize substrates which are relatively less expensive than those required for known processes.

Another object of the present invention to provide a process of gamma glutamulation to prepare gamma-glutamyl compounds that have relatively better organoleptic properties and taste as compared to their un-glutamylated parent compounds.

Another object of the present invention is to provide a process of gamma glutamylation to prepare gamma glutamyl compounds that have relatively high solubility as compared to their un-glutamylated parent compounds.

Another object of the present invention is to provide a process of gamma glutamylation to prepare gamma glutamyl compounds that have relatively better stability in the blood stream as compared to their un-glutamylated parent compounds.

The present inventors have surprisingly found that some or all of the foregoing objects can be achieved by using a gamma glutamyl transpeptidase enzyme derived from a plant material.

### SUMMARY OF THE INVENTION

According to the present invention there is provided a process for enzymatic preparation of gamma-glutamyl compound comprising a step of contacting a gamma-glutamyl donor and a gamma-glutamyl acceptor with an aqueous medium comprising a gamma glutamyl transpeptidase enzyme derived from a plant material where the plant belongs to Graminaceae or Leguminaceae family, or is Camellia sinensis.

According to a preferred aspect of the present invention, there is provided a process for enzymatic preparation of gamma-glutamyl compound wherein,
a. said donor is a peptide or an amino acid having gamma glutamyl moiety;
b. said acceptor is ethylamine or ethylamine hydrochloride, and;
c. said gamma glutamyl compound is theanine.

According to another aspect, there is provided a gamma-glutamyl compound obtainable by the process of the present invention.

According to a further aspect, there is provided a tea product comprising a gamma-glutamyl compound prepared by the process of the present invention.

### DETAILED DESCRIPTION OF THE INVENTION

### Process of Preparation of Gamma-Glutamyl Compound

According to the present invention there is provided a process for enzymatic preparation of gamma-glutamyl compound comprising a step of contacting a gamma-glutamyl donor and a gamma-glutamyl acceptor with an aqueous medium comprising a non-microbial gamma glutamyl transpeptidase derived from a plant material where the plant belongs to Graminaceae or Leguminaceae family, or is Camellia sinensis.

In the process of the present invention, an aqueous medium comprising GGT derived from a plant material is used.

The temperature of the aqueous medium is maintained preferably between 10 and 80° C., more preferably between 20 and 70° C., and most preferably between 25 and 60° C. during the process.

The pH of the aqueous medium is preferably less than 8.5, more preferably less than 8.0, more preferably still less than 7.5 and most preferably less than 7.0. Preferably the pH of the aqueous medium is at least 5.0, more preferably at least 5.5, more preferably still at least 6.0 and most preferably at least 6.5.

The step of contacting is for duration of preferably from 0.5 to 24 hours, more preferably from 12 to 24 hrs and most preferably from 12 to 16 hours.

At the end of the process, a reaction mixture is obtained. The reaction mixture comprises the gamma-glutamyl compound, water, unreacted substances and enzyme, if any. The reaction mixture is preferably further purified to separate the gamma-glutamyl compound. Thus in a preferred embodiment the gamma-glutamyl is separated from at least some of the solids in the reaction mixture.

### Gamma-Glutamyl Transpeptidase Enzyme

According to the process of the present invention, gamma-glutamyl transpeptidase (GGT) enzyme is derived from a plant material where the plant belongs to Graminaceae or Leguminaceae family, or is Camellia sinensis.

GGT is derived from a plant material obtained from any part of the plant including seed, fruit, bark, stem, callus, tuber, root, leaves, flower or a mixture thereof. Specific plant tissues, such as chloroplast, apoplast, mitochondria, vacuole, peroxisomes, and cytoplasm are also considered to be a plant material. Furthermore, any plant cell, whatever the tissue origin, is considered to be a plant material. Also included within the scope plant material are the progeny of such plants, plant parts and plant cells.

According to a preferred aspect, the enzyme has an activity greater than 50 units, more preferably greater than 200 units and most preferably greater than 500 units.

Enzyme activity is measured by the following method. 1 mg enzyme is added to a reaction mix containing 1 millimole of gamma glutamyl paranitro annilide and 400 millimoles of ethylamine hydrochloride and the mix is then incubated for 4 hrs at 25° C. The paranitroaniline released during the reaction is measured at 405 nm using spectrophotometer. The term enzyme activity as used herein is the amount of enzyme (mg) required to release 1 micromole of paranitroaniline under assay conditions given above.

The plant material is preferably a seed, and more preferably, a germinated seed. Germinated seed is the preferred plant material particularly when the plant belongs to Graminaceae or Leguminacea family. The term “germinated seed” as used herein means the seed in which the coleoptile and root tip has emerged from the embryo. For germinating, the seeds are preferably soaked in water for 2-3 hours. The wet seeds are preferably wrapped in a cotton cloth for 2-4 days till the coleoptile and root tips starts appearing from the embryo.

GGT is derived from a plant material where the plant belongs to Graminaceae or Leguminaceae family, or is Camellia sinensis. It is particularly preferred that the plant belongs to Graminaceae or Leguminaceae family. It is further preferred that the plant belongs to Graminaceae family.

Plant material from any plant belonging to Leguminaceae family can be used according to the present invention. Preferably, the plant belonging to Leguminacae family is selected from soya, pea, green gram, black gram, or mung bean. It is particularly preferred that the plant is selected from soya, green gram, black gram or mung bean. Most preferred is green gram.

Plant material from any plant belonging to Graminaceae family can be used according to the present invention. The plant belonging to Graminaceae family is preferably selected from maize, wheat, rice, barley, sorghum, triticale, rye, millet, buckwheat, fonia, or quinoa. More preferably the plant is sorghum, millet, wheat or rice. More preferably still the plant is sorghum or millet but especially millet. The millet variety that is particularly preferred is Eleusine coracana, which is also known as Ragi in India. Ragi, also known as finger millet, originated in Eastern Africa and was introduced to India nearly 3000 years ago. It has relatively low commercial value and is considered to be a “poor man's food”.

Method of Deriving GGT Enzyme from the Plant Material

GGT is preferably derived from the plant by a method comprising the steps of:
a. grinding the plant material;
b. extracting the ground plant material in an aqueous medium buffered at pH preferably between 5 and 11, more preferably between 6 and 10 most preferably between 6 and 8, thereby to produce a mixture of insoluble solids and supernatant crude protein extract;
c. separating insoluble solids from the mixture; and
d. recovering the supernatant crude protein extract comprising the GGT.

The crude extract comprising GGT may be used as such. It may also be further purified to obtain GGT-enriched fraction. Such a fraction may be prepared, for example, by subjecting the enzyme extract to ammonium sulphate precipitation (for example using up to 80% ammonium sulphate saturation).

According to a preferred aspect, The GGT enzyme has an optimum pH between 6.5 and 8.5.

### Gamma-Glutamyl Acceptor

The gamma-glutamyl acceptor according to the present invention is any compound that is capable of accepting a gamma-glutamyl moiety. The gamma-glutamyl acceptor is preferably selected from amine, amino acid, peptide or mixture thereof.

The amine is preferably a primary or secondary amine. Some examples of amines suitable for the present invention include, but are not limited to C1-C4 primary amines.

The amino acid is preferably aromatic, basic or branched amino acid. Preferably, the amino acid is proteinogenic amino acid or non-protein amino acid. The proteinogenic amino acid is preferably selected from glutamine, glutamic acid, tryptophan, valine, leucine, lysine, phenylalanine or tyrosine. The non-protein amino acid is preferably selected from L-dopa or taurine.

The peptide is preferably selected from glycylglycine or cystinylglycine.

### Gamma-Glutamyl Donor

The term gamma-glutamyl donor as used herein means any compound having a gamma-glutamyl moiety. Gamma-glutamyl donor is preferably selected from an amino acid, a peptide or derivative thereof.

The preferred gamma-glutamyl donor according to the present invention is selected from glutathione, glutamine, gamma-glutamyl alanine, gamma-glutamyl tryptophane, gamma-glutamyl cysteine, gamma-glutamyl valine, gamma-glutamyl histidine, or gamma glutamyl leucine.

### Gamma-Glutamyl Compounds

The process according to the present invention can be used for preparation of many types of gamma-glutamyl compounds. The process of gamma glutamylation can improve taste and organoleptic properties of certain amino acids and peptides, particularly those with bitter taste.

### Theanine

L-theanine (Gamma-glutamyl ethyl amide), can be prepared according to the process of the present invention.

According to one aspect of the present invention, there is provided a process for preparation of a gamma-glutamyl compound wherein,
a. said gamma-glutamyl donor is a peptide or an amino acid having gamma glutamyl moiety;
b. said gamma-glutamylacceptor is ethylamine or ethylamine hydrochloride; and
c. said gamma glutamyl compound is theanine.

At the end of the step of contacting, a reaction mixture comprising theanine, unreacted gamma-glutamyl donor, unreacted gamma-glutamyl acceptor, unreacted enzyme, if any and water is obtained.

According to a preferred aspect, the process comprises a further step of purification of theanine by extraction with water followed by separation of insoluble solids to obtain an aqueous solution comprising theanine.

Preferably, the aqueous solution is dried to prepare a solid comprising theanine.

According to a particularly preferred aspect of the present invention, the source of said donor is a protein hydrolysate. Protein hydrolysate is preferably selected from whey protein hydrolysate, soya protein hydrolysate, gluten protein hydrolyasate or mixture thereof.

It will be appreciated that protein hydrolysate as a source of donor of gamma-glutamyl moiety offers significant economic advantage as compared to prior art donors such as glutathione, gamma-glutamyl paranitro annilide, glutamyl cysteine, glutamyl tryptophan etc.

Commercially available protein hydrolysate is typically a mixture of hydrolysed protein as well as unhydrolysed protein, and can be used according to the present invention. The hydrolysed protein is about 25-35% whilst unhydrolysed protein is about 65-75%.

The protein hydrolysate comprises preferably less than 60%, more preferably less than 30% and most preferably less than 5% unhydrolysed protein. It is particularly preferred that the protein hydrolysate is substantially free from the unhydrolysed protein.

One of the ways in which the extent of unhydrolysed protein can be reduced from protein hydrolysate is by alcohol precipitation. Typically C2-C5 alcohol is added to the protein hydrolysate and the resulting mix is kept at 4° C. for 12-14 hours. The mix is then centrifuged and the supernatant is lyophilized and used as a source of said donor. Thus, according to a preferred aspect, protein hydrolysate is subjected to at least 75% C2-C5 alcohol saturation.

Without wishing to be limited by theory, it is believed that the process of alcohol saturation improves the yield of theanine as the unhydrolysed protein, which can act as inhibitor for GGT, is removed. Further, since the unhydrolysed protein is removed, organoleptic properties are not negatively affected by presence of unhydrolysed protein.

Tea Product with Enhanced Theanine

According to a further preferred aspect, the aqueous solution comprising theanine or the solid comprising theanine is added to a tea material to obtain a tea product with enhanced theanine. When aqueous solution comprising theanine is added to a tea material, the tea material may be further subjected to a step of drying to reduce the moisture content below 5% by weight of the tea product.

According to one aspect, there is provided a tea product comprising a gamma-glutamyl compound prepared by the process of the present invention.

According to another aspect, there is a provided a tea product with enhanced theanine prepared by addition to a tea product of theanine prepared by process according to the present invention.

### EXAMPLES

The invention will now be demonstrated with examples. The examples are by way of illustration only and do not limit the scope of the invention in any manner.

### Materials and Methods

### Materials

Tris Base, Glutathione, gamma glymayl histidine, gamma glutamyl alanine, gamma glutamyl tryptophane, gamma glutamyl valine, glycyl glycine, O-Pthalaldehyde (OPA) and Pentadecafluorooctanoic Acid (98%) were obtained from Sigma chemicals. Ethylamine hydrochloride was obtained from Fluka. Methanol (AR grade), Brij 35 (30% w/v), and acetonitrile (HPLC grade) were obtained from Merck. 2-Mercaptoethanol, Boric acid (AR grade), Potassium hydroxide, (AR grade), and Ethanol (AR grade) were obtained from SRL, India. Theanine (Suntheanine™) was procured from Tayo.

Seeds of Ragi, maize, peas, green gram, and sorghum were purchased from local market in Bangalore, India. Tea seeds were procured from Tea gardens in South India.

### Methods

### Method for Assay of Enzyme

GGT assay was carried out according to Storozhenko et al. (Plant Physiology, 128:1109-1119, 2002). In brief, 1 mg of enzyme was added to a reaction mix containing 1 milli mole of gamma glutamyl paranitroanaylide and 400 millimoles of ethylamine hydrochloride and incubated for 4 hours at 25° C. The paranitroaniline released through during the reaction was measured at a wavelength of 405 nm using a spectrophotometer. Enzyme activity is defined as the amount of enzyme required to release 1 micromole of paranitroaniline under assay conditions.

### Assay of Theanine (HPLC)

Theanine assay was carried out by adding 500 μg of enzyme to the reaction mix containing varied concentration of gamma glutamyl donor and varied concentration of ethylamine hydrochloride in a buffer (pH ranging from 5.5 to 11.5). 20 μL reaction mix was injected into HPLC.

Theanine was detected fluorimetrically following post-column derivatisation with o-Pthalaldehyde. The elutant from the column is fed into a low dead-volume 3-way junction and mixed with the o-Pthalaldehyde reagent in a 1:1 ratio, the o-Pthalaldehyde reagent being pumped at 1 ml/minute by the isocratic pump. In the detector, excitation was at a wavelength of 340 nm and emission was at a wavelength of 425 nm.

Method of Deriving GGT from Plant Material

The procedure below is described with reference to Ragi (Eleusine coracana). A similar procedure was used for other plants including maize, sorghum, peas, green gram and tea.

(a) Seeds of Ragi (Eleusine coracana)_were germinated for 4 days.

(b) 50 g of germinated seed material was ground and mixed with 250 mL of high saline buffer (buffer preparation as described by Shaw et al., Phytochemistry 66 515-522, 2005) and polyvinyl pyrollidone phosphate (2%) to remove polyphenols.

(c) The mixture was stirred for 1 hour whilst the temperature was maintained at 4° C.

(d) The mixture was then centrifuged at rpm of 10000 for 30 minutes. The supernatant was separated and used for further separation.

(e) Ammonium sulphate was added to the supernatant and the resulting mixture was centrifuged. The solid pellet containing the enzyme fraction was separated from the supernatant.

(f) Step (e) was repeated with the supernatant obtained at the end of step (e) to obtain a second solid pellet comprising enzyme fraction.

(g) The solid pellets obtained in steps (e) and (f) were mixed together.

30-70% ammonium sulfate fraction of the crude protein extract was used in the experiments. It represents the protein that does not get precipitated with 30% ammonium sulfate and gets precipitated with 70% ammounium sulfate in a sequential manner.

### Experimental Protocol

Experiments were carried out using glutathione and soya protein hydrolysate as donors of gamma-glutamyl moiety. Soya protein hydrolysate was used at 0.5-20% by weight per unit volume of the total reaction mixture. Ethylamine hydrochloride was used as an acceptor of gamma-glutamyl moiety. Crude enzyme extract comprising GGT derived from Ragi was used in the experiments. The mass of enzyme used was 500 μg (30-70% fraction) unless specified otherwise. Total volume was made up to 1 mL with various buffers (pH ranging from 5.5 to 11.5). Reaction mixture was incubated at 40° C. for 4 hours, unless specified otherwise. After incubation for the duration required, the reaction was stopped by adding 200 μL of 2 N acetic acid.

### Effect of pH on Theanine Yield

Source of gamma-glutamyl donor was soya protein hydrolysate (1% wt per unit volume of reaction mixture). Acceptor of gamma-glutamyl moiety was ethylamine hydrochloride at 400 mM. The temperature of incubation was 25° C. and duration of incubation was 4 hours. The pH was varied from 5.5 to 11.5 by using buffers as given in Table 1. Theanine was measured by HPLC. The results are given in Table 1.

TABLE 1

| Example | | | Theanine |
|---|---|---|---|
| No | pH | Buffer used | (μg/mL) |
| 1 | 5.5 | Citrate-phosphate | 4.2 |
| 2 | 6.5 | Citrate-phosphate | 7.4 |
| 3 | 7.5 | Tris (hydroxymethyl) | 7.8 |
| | | aminomethane | |
| 4 | 8.5 | Tris (hydroxymethyl) | 7.0 |
| | | aminomethane | |
| 5 | 9.5 | Carbonate-bicarbonate | 5.2 |
| 6 | 10.5 | Carbonate-bicarbonate | 4.4 |
| 7 | 11.5 | Carbonate-bicarbonate | 4.2 |

From the above results, it is clear that preparation of theanine can be carried out over a broad range of pH using the enzyme obtained from plant material. Further, the pH optimum of the GGT enzyme obtained from the plant material is between 6.5 and 8.5. The activity at pH of 6.5 is about 95% of the maximum activity. In comparison, GGT obtained from animal or microbial source has a pH optimum at about 9.5, and can not be used over a broad range of pH. Although GGT obtained from radish was reported to have a pH optimum between 7.5-8, the activity was reported to drop to 50% at a pH of 7.

### Effect of Temperature

Source of gamma-glutamyl donor was soya protein hydrolysate (1% wt per unit volume of reaction mixture). Acceptor of gamma-glutamyl moiety was ethylamine hydrochloride at 400 mM. The duration of incubation was 4 hours, and the pH was maintained at 7.5. Theanine, as measured by HPLC, is given in Table 2.

TABLE 2

| Example No | Temperature (° C.) | Theanine (μg/mL) |
|---|---|---|
| 8 | 25 | 4.1 |
| 9 | 40 | 7.9 |
| 10 | 50 | 7.9 |
| 11 | 60 | 6.5 |
| 12 | 70 | 4.5 |

From the above results, it is clear that preparation of theanine can be carried out over a broad range of temperature using the enzyme obtained from plant material.

### Effect of Amount or Protein Hydrolysate

Source of gamma-glutamyl donor was soya protein hydrolysate at various amounts. Acceptor of gamma-glutamyl moiety was ethylamine hydrochloride at 400 mM. The temperature of incubation was 40° C., the pH was maintained at 7.5, and duration of incubation was 4 hours. Results are given in Table 3.

TABLE 3

| | Amount of protein hydrolysate | |
|---|---|---|
| Example | (% wt per mL of reaction | Theanine |
| No | mixture) | (μg/mL) |
| 13 | 0.5 | 5.2 |
| 14 | 1 | 9.2 |
| 15 | 2 | 15.5 |
| 16 | 5 | 29.7 |
| 17 | 10 | 90.7 |
| 18 | 20 | 118.8 |

From the above results, it is clear that yield of theanine increases with the amount of protein hydrolysate in the range of amount of protein hydrolysate used. It will be appreciated that protein hydrolysate is a relatively cheap and readily available source of gamma-glutamyl donor that provides relatively high yield of theanine.

### Effect of Duration of Incubation

Source of gamma-glutamyl donor was soya protein hydrolysate (10% we per unit volume of reaction mixture). Acceptor of gamma-glutamyl moiety was ethylamine hydrochloride at 10 mM. The temperature of incubation was 40° C., pH was maintained at 7.5, and duration of incubation was 4 hours. Results are given in Table 4.

TABLE 4

| | Duration of | Theanine |
|---|---|---|
| Example No | incubation (hours) | (μg/mL) |
| 19 | 2 | 21 |
| 20 | 4 | 42 |
| 21 | 6 | 68 |
| 22 | 8 | 134 |
| 23 | 10 | 148 |
| 24 | 12 | 463 |
| 25 | 16 | 164 |
| 26 | 22 | 162 |

Duration of incubation from about 8 to 22 hours provides relatively higher yields of theanine. The highest yield of theanine is obtained when the duration of incubation is between 10 to 16 hours, in particular about 12 hours.

Enzyme Activity from Various Plant Materials

Crude enzyme extract (30-70%) was obtained from germinated seeds of various plants according to the procedure described earlier. In case of tea, the enzyme extract was obtained from germinated seed as well as leaf. The enzyme extract was assayed for GGT activity spectrophotometrically and the results are given in Table 5.

TABLE 5

| Plant | GGT enzyme activity |
|---|---|
| Ragi (Eleusine coracana) seed | 1532.8 |
| Maize seed | 233.6 |
| Sorghum seed | 321.2 |
| Green gram seed | 349.2 |
| Pea seed | 62.8 |
| Tea seed | Not detected |
| Tea leaf | 244.9 |

It is clear that crude protein extract obtained from all the plants including cereal grains, and legume has relatively high GGT enzyme activity. In case of tea, crude protein extract obtained from seed material does not have detectable gamma-glutamyl activity. However, crude protein extract obtained from tea leaf shows relatively high gamma-glutamyl activity. Further, it is evident that crude protein extract from Ragi (Eleusine coracana) in particular gives a surprisingly high GGT enzyme activity.

### Glutathione as Donor of Gamma-Glutamyl Moiety

Acceptor of gamma-glutamyl moiety was ethylamine hydrochloride at 400 mM. Crude enzyme extract comprising GGT derived from Ragi was used in the experiments. The mass of enzyme used was 250 μg. The temperature of incubation was 25° C., pH was maintained at 9.5, and duration of incubation was 4 hours. Theanine, as measured by HPLC, was found to be 22.7 μg/mL, indicating that the process of the present invention can be used with various donors of gamma-glutamyl moiety.

### Effect of Alcohol Precipitation on Theanine Yield

Acceptor of gamma-glutamyl moiety was ethylamine hydrochloride at 10 mM. The temperature of incubation was 40° C., the pH was maintained at 7.5, and duration of incubation was 16 hours. 1 mg/mL GGT crude extract obtained from Ragi was used. The experiments were carried out with source of gamma-glutamyl donor as soya protein hydrolysate as commercially obtained. Experiments were also carried out with supernatant obtained after 75% ethanol precipitation of soya protein hydrolysate to remove unhydrolysed protein.

It was found that the yield of theanine was 158 μg/mL with soya protein hydrolysate as commercially obtained (which comprised about 65-75% unhydrolysed protein). The protein hydrolysate from which the unhydrolysed protein was removed by alcohol precipitation surprisingly gave a higher yield of theanine (207 μg/mL).

It will be appreciated that the process of the present invention can be carried out to prepare gamma-glutamyl compound, in particular, theanine, over a relatively broad range of pH and temperature. Moreover, the process can be used at pH less than 8.5, and even at a pH of 6.5 without significant decrease in the enzyme activity. Further, the process of the present invention is of economic significance as it allows use of relatively less expensive materials.

1. A process for the enzymatic preparation of a gamma-glutamyl compound, the process comprising a step of contacting a gamma-glutamyl donor and a gamma-glutamyl acceptor with an aqueous medium comprising a gamma glutamyl transpeptidase enzyme derived from a plant material where the plant belongs to Graminaceae or Leguminaceae family, or is Camellia sinensis.

2. A process as claimed in claim 1 wherein the pH of the aqueous medium is less than 8.5, preferably less than 8.0.

3. A process as claimed in claim 2 wherein the pH is between 6.0 and 8.5.

4. A process as claimed in claim 3 wherein the pH is between 6.5 and 8.0.

5. A process as claimed in claim 1 wherein said enzyme has optimum activity at a pH between 6.5 and 8.5.

6. A process as claimed in claim 1 wherein the enzyme is derived by a method comprising the steps of:
a. grinding the plant material;
b. extracting the ground plant material with an aqueous medium buffered at pH between 5 and 11, thereby to produce a mixture of insoluble solids and supernatant crude protein extract;
c. separating insoluble solids from the mixture; and
d. recovering supernatant crude protein extract comprising said enzyme.

7. A process as claimed in claim 1 wherein:
(a) the plant belongs to Graminaceae family and is selected from sorghum, millet, wheat, rice or
(b) the plant belongs to Leguminaceae family and is selected from soya, green gram, black gram or mung bean.

8. A process as claimed in claim 7 wherein the plant is selected from sorghum, millet or green gram.

9. A process as claimed in claim 8 wherein said plant is millet.

10. A process as claimed in claim 9 wherein said millet is Eleusine coracana.

11. A process as claimed in claim 1 wherein said acceptor is selected from amine, amino acid, peptide or mixture thereof.

12. A process as claimed in claim 11 wherein said amino acid is proteinogenic amino acid or non-protein amino acid.

13. A process as claimed in claim 12 wherein said proteinogenic amino acid is selected from glutamine, glutamic acid, tryptophan, valine, leucine, lysine, phenylalanine or tyrosine.

14. A process as claimed in claim 1 wherein the gamma-glutamyl donor is selected from an amino acid, peptide or derivative thereof.

15. A process as claimed in claim 1 wherein said enzyme has an activity greater than 50 units.

16. A process as claimed in claim 1 wherein,
a. said donor is a peptide or an amino acid having gamma glutamyl moiety,
b. said acceptor is ethylamine or ethylamine hydrochloride, and;
c. said gamma glutamyl compound is theanine.

17. A process as claimed in claim 16 wherein source of said donor is a protein hydrolysate.

18. A process as claimed in claim 17 wherein said protein hydrolysate is selected from whey protein hydrolysate, soya protein hydrolysate, gluten protein hydrolysate, or mixture thereof.

19. A process as claimed in claim 1 comprising a further step of purification of theanine by extraction with water followed by separation of insoluble solids to obtain an aqueous solution comprising theanine.

20. A process as claimed in claim 19 wherein said aqueous solution is dried to obtain a solid comprising theanine.

21. A process as claimed in claim 1 wherein said aqueous solution or said solid is contacted with a tea material, followed by optionally drying the material, to obtain a tea product with enhanced theanine.

22. A gamma-glutamyl compound obtainable by the process as claimed in claim 1.

23. A tea product comprising a gamma-glutamyl compound as claimed in claim 22.
